(19) United States
(12) Patent Application Publication
(10) Pub. No.: US 2022/0004885 A1
(43) Pub. Date: Jan. 6, 2022

(54) COMPUTER SYSTEM AND CONTRIBUTION CALCULATION METHOD

(71) Applicant: HITACHI, LTD., Tokyo (JP)

(72) Inventors: Haruka YAMADA, Tokyo (JP); Naoaki YOKOI, Tokyo (JP); Masashi EGI, Tokyo (JP)

(73) Assignee: HITACHI, LTD., Tokyo (JP)

(21) Appl. No.: 17/206,787

(22) Filed: Mar. 19, 2021

(30) Foreign Application Priority Data
Jul. 2, 2020 (JP) 2020-115117

(51) Int. Cl. G06N 5/02

(52) U.S. Cl. CPC G06N 5/02

(57) ABSTRACT

A computer system includes a calculation unit for extracting specific reference data from a plurality of reference data, configured to calculate a contribution of the each feature amount of explanatory data regarding a predicted value using the specific piece of reference data, the explanatory data, and a predictor, and stores the contribution that has been calculated as a pair contribution in association with the specific piece of the reference data and the explanatory data, the pair contribution being a contribution that has been calculated with the one piece of the reference data and the explanatory data being a pair, for all pairs including each reference data and the explanatory data; and an aggregation unit for reading the pair contribution that has been calculated for the each feature amount of the explanatory data, and configured to calculate by aggregating the contribution of the each feature amount of the explanatory data.

### BACKGROUND OF THE INVENTION

### 1. Field of the Invention

The present invention generally relates to a calculation of a contribution of each feature amount in explanatory data with respect to a predicted value of the explanatory data.

### 2. Description of the Related Art

In these years, Artificial Intelligence (AI) is increasingly becoming a black box, and this makes it difficult to interpret grounds that have been determined by the AI (determination grounds). For the reasons of transparency, fairness, and the like of the determinations made by the AI, disclosure of the determination grounds of the AI is socially demanded, and Explainable AI (XAI) technologies attract attention.

SHapley Additive exPlanations (SHAP) is one of the XAI technologies. According to the SHAP, it can be understood how much each feature amount of certain data X has a positive or negative effect on a predicted value of the data X. However, in a case where the SHAP is used, only obvious explanations are given in some cases.

For example, in a mortality risk prediction in the medical field, assuming that a predicted value of an elderly person X is 80%. The explanation by the SHAP is that “the contributions of age-related features are high”. In other words, the SHAP explains that the high mortality risk results from an old age. In the SHAP calculation, reference data is set (generally all teacher data is set), and the SHAP value (an example of contribution) of each feature amount of the data (explanatory data) of the elderly person X is calculated using all reference data as a reference. Hence, only obvious explanations are given in many cases.

In this regard, H. Chen, “Explaining Models by Propagating Shapley Values”, 2019 proposes limiting the reference data. For example, in calculating the SHAP value by limiting the reference data to elderly people similar to the elderly person X, it is found that, for example, in particular, among the elderly people, “blood pressure” increases the mortality risk of the elderly person X.

In a case where the technology described in H. Chen, “Explaining Models by Propagating Shapley Values”, 2019 is utilized, it can be assumed that a user conducts recalculations of the SHAP values by limiting the reference data while interacting with the elderly person X who is a customer, such that what will happen when too much alcohol drinking is used as the reference, what will happen when male is used as the reference, and the like.

### SUMMARY OF THE INVENTION

In an actual case, however, for example, there is a large number of the reference data, and a recalculation of the SHAP value by limiting the reference data needs a long calculation time. In other words, it takes time to recalculate the SHAP value due to a change of the reference data, and therefore a user is not able to communicate with the customer in a smooth manner.

The present invention has been made in consideration of the above circumstances, and proposes a computer system and the like capable of appropriately providing a contribution of each feature amount of explanatory data.

In order to address such an issue, in the present invention, provided is a computer system that uses a predictor configured to conduct a prediction, explanatory data that is data to be a prediction target of the predictor, and a plurality of pieces of reference data that are data to be used as a reference in comparison with the explanatory data, and that calculates a contribution of each feature amount of the explanatory data with respect to a predicted value of the explanatory data that has been predicted by the predictor, the computer system including: a calculation unit configured to extract one piece of the reference data from the plurality of pieces of reference data, configured to calculate the contribution of each feature amount of the explanatory data with respect to the predicted value by using the one piece of the reference data, the explanatory data, and the predictor, and configured to store, in a storage device, the contribution that has been calculated as a pair contribution in association with the one piece of the reference data and the explanatory data, the pair contribution being a contribution that has been calculated with the one piece of the reference data and the explanatory data being a pair, for all pairs including each reference data of the plurality of pieces of reference data and the explanatory data; and an aggregation unit configured to read, from the storage device, the pair contribution that has been calculated by the calculation unit for the each feature amount of the explanatory data, and configured to calculate by aggregating the contribution of the each feature amount of the explanatory data.

In the above configuration, the pair contribution that has been calculated with each reference data as a reference is stored in the storage device. For example, according to the above configuration, the aggregation unit is capable of reading the pair contribution from the storage device, and aggregating the pair contribution. Therefore, the contribution of each feature amount of the explanatory data can be output in a prompt manner, according to a change of a reference condition.

According to the present invention, a computer system that is high in convenience can be realized.

### BRIEF DESCRIPTION OF THE DRAWINGS

FIG. 1 is a diagram showing an example of a configuration related to a computer system according to a first embodiment;

FIG. 2 is a diagram showing an example of a configuration of a computer according to the first embodiment;

FIG. 3 is a diagram showing an example of a reference data DB according to the first embodiment;

FIG. 4 is a diagram showing an example of a contribution data DB according to the first embodiment;

FIG. 5 is a diagram showing an example of a cluster data DB according to the first embodiment;

FIG. 6 is a diagram showing an example of a characteristic configuration of the computer system according to the first embodiment;

FIG. 7 is a diagram showing an example of the characteristic configuration of the computer system according to the first embodiment;

FIG. 8 is a diagram showing an example of the characteristic configuration of the computer system according to the first embodiment;

FIG. 9 is a diagram showing an example of the characteristic configuration of the computer system according to the first embodiment;

FIG. 10 is a diagram showing an example of a contribution explanation screen according to the first embodiment;

FIG. 11 is a diagram showing an example of a reference change screen according to the first embodiment;

FIG. 12 is a diagram showing an example of a cluster setting screen according to the first embodiment;

FIG. 13 is a diagram showing an example of a process performed by a mutual calculation unit according to the first embodiment;

FIG. 14 is a diagram showing an example of a process performed by a calculation unit according to the first embodiment;

FIG. 15 is a diagram showing an example of a process performed by an aggregation unit according to the first embodiment;

FIG. 16 is a diagram showing an example of a process performed by a search unit according to the first embodiment;

FIG. 17 is a diagram showing an example of a process performed by a similarity calculation unit according to the first embodiment;

FIG. 18 is a diagram showing an example of a process performed by a cluster generation unit according to the first embodiment;

FIG. 19 is a diagram showing an example of a process performed by a cluster output unit according to the first embodiment; and

FIG. 20 is a diagram showing an example of a process performed by the cluster output unit according to the first embodiment.

### DESCRIPTION OF THE PREFERRED EMBODIMENTS

### (1) First Embodiment

Hereinafter, an embodiment of the present invention will be described in detail. In the present embodiment, a description will be given with regard to a calculation of a contribution of each feature amount in explanatory data with respect to a predicted value of the explanatory data using a predictor (a machine learning model). However, the present invention is not limited to the embodiment.

In a computer system in the present embodiment, every record is selected from R records of the reference data, the contributions (for example, SHAP values) of the R records are calculated using only each one record as a new reference data, and a calculation result is stored as a pair contribution. At the first time, the calculation results stored beforehand are averaged for each feature amount, and the average is output. At the second and subsequent times, the pair contributions that have been calculated beforehand using the limited R′ records of the reference data as the respective references are searched for and aggregated, and an aggregation result is output.

As a technique for interpreting a predicted value that has been predicted by the predictor, various tools for analyzing a prediction result with respect to the data by giving a perturbation have been devised, such as SHAP and local interpretable model-agnostic explanations (LIME). The present invention is applicable to various tools that use perturbation analysis.

Next, an embodiment of the present invention will be described with reference to the drawings.

It is to be noted that in the following description, the same elements will be assigned with the same numerals in the drawings, and the description will be omitted as appropriate. In addition, in a case where a description is given without distinguishing between elements of the same type, a common part (a part excluding a branch number) out of reference numerals including branch numbers is used, whereas in describing by distinguishing the elements of the same type, a reference numeral including a branch number is used in some cases. For example, in a case where a description is given without distinguishing between computers in particular, “computer 100” is used, whereas in a case where a description is given by distinguishing between individual computers, “computer 100-1” and “computer 100-2” are used in some cases.

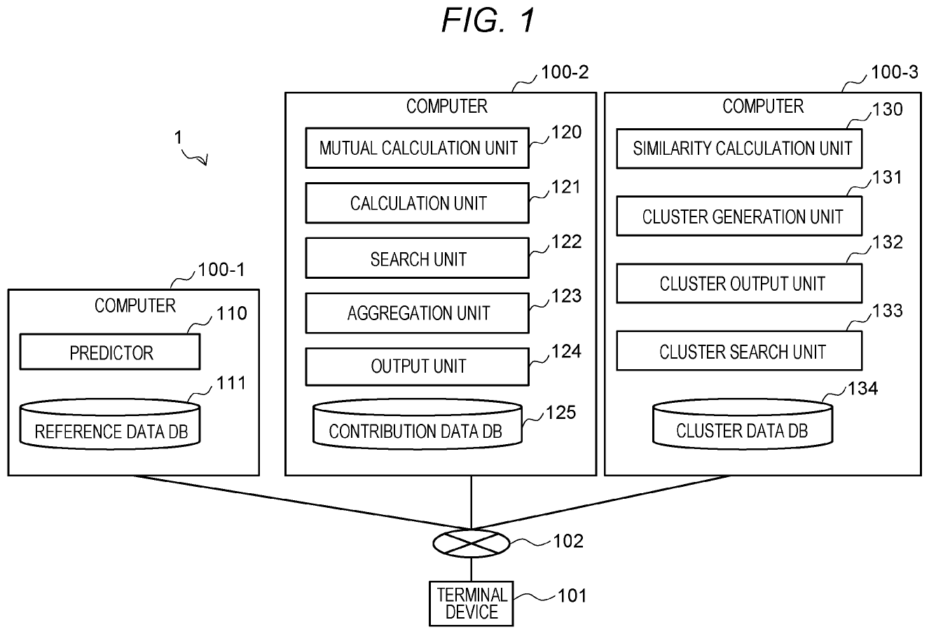

In FIG. 1, reference numeral 1 denotes a computer system as a whole, according to a first embodiment.

FIG. 1 is a diagram showing an example of a configuration related to the computer system 1.

In the computer system 1, for example, data (explanatory data) to be predicted (risk diagnosis, object detection, and the like) is input, the explanatory data is predicted, a contribution of each feature amount of the explanatory data is calculated, and a predicted value, which is a result of the prediction, and the contribution of each feature amount of the explanatory data are output.

The computer system 1 includes one or more computers 100 and one or more terminal devices 101. The computer 100 and the terminal device 101 are communicably coupled to each other via a network 102.

A computer 100-1 includes a predictor 110 and a reference data DB 111. The predictor 110 is a machine learning model, and predicts the explanatory data that has been input by the terminal device 101. The reference data DB 111 stores a plurality of reference data. The reference data is data that can be used as a reference in the calculation of a contribution of each feature amount of the explanatory data. The reference data may be teacher data of the predictor 110, test data of the predictor 110, data that have been input by a user in an operation of the computer system 1, any combination of the above data, or any other data.

A computer 100-2 includes a mutual calculation unit 120, a calculation unit 121, a search unit 122, an aggregation unit 123, an output unit 124, and a contribution data DB 125.

The mutual calculation unit 120 selects a pair of two records (a pair including one record used as explanatory data and the other one record used as reference data) from the reference data DB 111, and calculates a contribution using the predictor 110 for all pairs. The contribution is a value indicating how much each feature amount of the explanatory data has an influence on the prediction of the explanatory data. The contribution that has been calculated is stored in the contribution data DB 125, in a case where one record of the reference data is used as a reference, as a pair contribution (contribution data) indicating a contribution of the explanatory data (the other one record of the reference data).

The calculation unit 121 selects a pair including the explanatory data that has been input into the terminal device 101 and one reference data in the reference data DB 111, and calculates a contribution using the predictor 110 for all the pairs. The contribution that has been calculated is stored in the contribution data DB 125 as a pair contribution (contribution data) indicating the contribution of the explanatory data, in a case where one record of the reference data is used as a reference.

The search unit 122 searches the contribution data DB 125 for the pair contribution corresponding to the reference data and the explanatory data that satisfy a reference condition to be described later. The aggregation unit 123 aggregates the pair contribution that has been searched for by the search unit 122 with respect to the respective feature amounts of the explanatory data, and sets the contribution that has been aggregated to a contribution of each feature amount of the explanatory data. The output unit 124 outputs the contribution that has been aggregated by the aggregation unit 123.

A computer 100-3 includes a similarity calculation unit 130, a cluster generation unit 131, a cluster output unit 132, a cluster search unit 133, and a cluster data DB 134.

The similarity calculation unit 130 calculates a similarity between the data (a similarity between one record of the explanatory data and one record of the reference data and a similarity between records of the reference data), based on contribution data stored in the contribution data DB 125. The cluster generation unit 131 generates a cluster based on the similarity that has been calculated by the similarity calculation unit 130. It is to be noted that a clustering method is not specified in particular. Hereinafter, hierarchical clustering will be described as an example. The data related to the cluster that has been generated by the cluster generation unit 131 is stored in the cluster data DB 134.

The cluster output unit 132 outputs information related to the cluster that has been generated by the cluster generation unit 131. The cluster search unit 133 refers to the cluster data DB 134, and searches for the cluster to which the explanatory data belongs.

The terminal device 101 inputs data, outputs data, sends data to the computer 100, and receives data from the computer 100. For example, the terminal device 101 sends, to the computer 100-2, the explanatory data, with which a prediction is requested by a user. Further, for example, the terminal device 101 displays a predicted value that has been calculated by the computer 100-2 and a contribution of each feature amount of the explanatory data. Further, for example, the terminal device 101 displays information of the cluster to which the explanatory data that has been calculated by the computer 100-3 belongs.

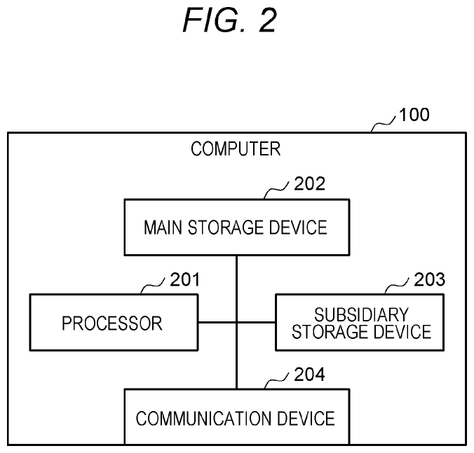

FIG. 2 is a diagram showing an example of a configuration of the computer 100.

The computer 100 is a server device, a notebook computer, a tablet terminal, or the like. The computer 100 includes a processor 201, a main storage device 202, a subsidiary storage device 203, and a communication device 204.

The processor 201 is a device that performs arithmetic processes. The processor 201 is, for example, a CPU (Central processing Unit), an MPU (Micro processing Unit), a GPU (Graphics processing Unit), an AI (Artificial Intelligence) chip, or the like.

The main storage device 202 is a device that stores programs, data, and the like. The main storage device 202 is, for example, a ROM (Read Only Memory), a RAM (Random Access Memory), or the like. The ROM is an SRAM (Static Random Access Memory), a NVRAM (Non Volatile RAM), a mask ROM (Mask Read Only Memory), a PROM (Programmable ROM), or the like. The RAM is a DRAM (Dynamic Random Access Memory) or the like.

The subsidiary storage device 203 is an HDD (Hard Disk Drive), an FM (Flash Memory), an SSD (Solid State Drive), an optical storage device, or the like. The optical storage device is a CD (Compact Disc), a DVD (Digital Versatile Disc), or the like. Programs, data, and the like stored in the subsidiary storage device 203 are read into the main storage device 202 when necessary.

The communication device 204 is a communication interface that communicates with another computer via a communication medium. The communication device 204 is, for example, an NIC (Network Interface Card), a wireless communication module, a USB (Universal Serial Interface) module, a serial communication module, or the like. The communication device 204 can also function as an input device that receives information from another computer that is communicably coupled. In addition, the communication device 204 can also function as an output device that sends information to another computer that is communicably coupled.

The computer 100 may include an input device, an output device, and the like. The input device is a user interface that receives information from a user. The input device is, for example, a keyboard, a mouse, a card reader, a touch panel, or the like. The output device is a user interface that outputs various information (display output, audio output, print output, and the like). The output device is, for example, a display device that visualizes various information, an audio output device (speaker), a printing device, and the like. The display device is an LCD (Liquid Crystal Display), a graphic card, or the like.

Functions of the computer 100 (the mutual calculation unit 120, the calculation unit 121, the search unit 122, the aggregation unit 123, the output unit 124, the contribution data DB 125, the similarity calculation unit 130, the cluster generation unit 131, the cluster output unit 132, the cluster search unit 133, the cluster data DB 134, and the like) may be realized by, for example, the processor 201 reading a program stored in the subsidiary storage device 203 into the main storage device 202 and executing the program (software), may be realized by hardware such as a dedicated circuit or the like, or may be realized by combining software and hardware.

It is to be noted that one function of the computer 100 may be divided into a plurality of functions, or the plurality of functions may be combined into one function. Further, a part of the functions of the computer 100 may be provided as another function, or may be included in another function. Further, a part of the functions of the computer 100 may be realized by another computer capable of communicating with the computer 100.

It is to be noted that the terminal device 101 is a personal computer, a notebook computer, a tablet terminal, or the like. The configuration of the terminal device 101 is identical or similar to that of the computer 100. Therefore, the description will be omitted.

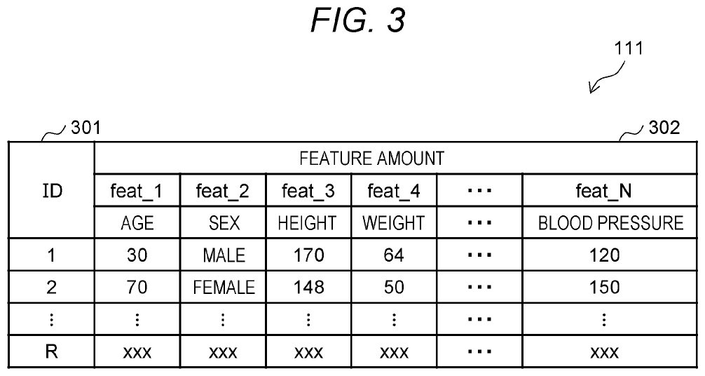

FIG. 3 is a diagram showing an example of the reference data DB 111.

The reference data DB 111 stores the reference data. More specifically, the reference data DB 111 stores a record in which an ID 301 and a feature amount 302 are associated with each other. The ID 301 is an ID for identifying the reference data. The feature amount 302 includes data of each feature amount (for example, each data item) of the reference data.

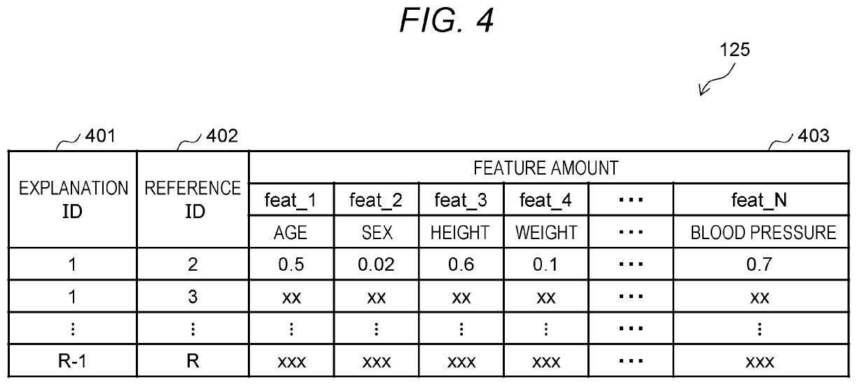

FIG. 4 is a diagram showing an example of the contribution data DB 125.

The contribution data DB 125 stores contribution data. More specifically, the contribution data DB 125 stores a record (a contribution vector) in which an explanation ID 401, a reference ID 402, and a feature amount 403 are associated with one another. The explanation ID 401 is an ID that can identify explanatory data. The reference ID 402 is an ID that can identify reference data. The feature amount 403 includes data of a contribution of each feature amount in the explanatory data.

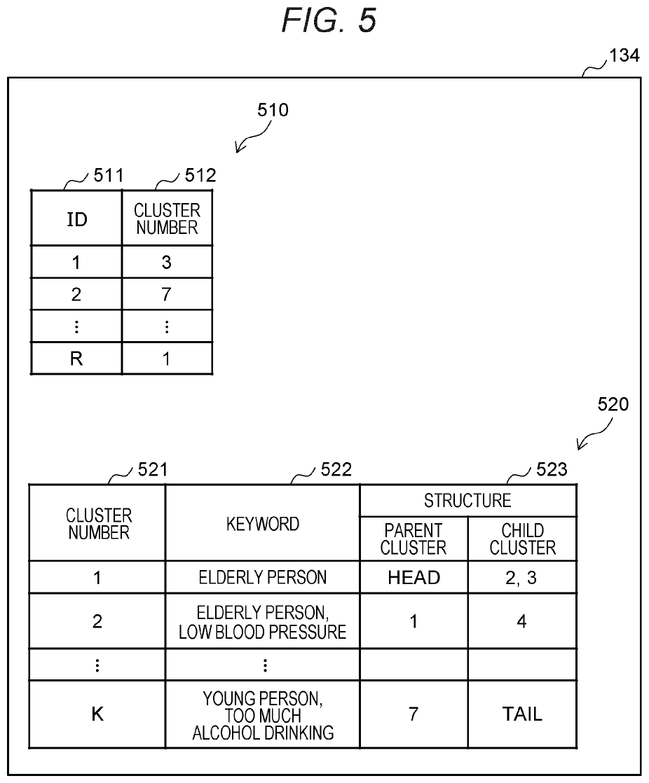

FIG. 5 is a diagram showing an example of the cluster data DB 134.

The cluster data DB 134 stores data related to the cluster. More specifically, the cluster data DB 134 is configured to include a cluster belonging table 510 and a cluster structure table 520.

The cluster belonging table 510 stores data that can identify a cluster to which the explanatory data and the reference data belong. More specifically, the cluster belonging table 510 stores a record in which an ID 511 and a cluster number 512 are associated with each other. The ID 511 is an ID that can identify explanatory data or an ID that can identify reference data. The cluster number 512 is a number that can identify a cluster.

The cluster structure table 520 stores a record in which a cluster number 521, a keyword 522, and a structure 523 are associated with each other. The cluster number 521 is a number that can identify a cluster. The keyword 522 is a keyword (name) for indicating a cluster. For example, in a case of a cluster having a hierarchical structure, the structure 523 includes data indicating the hierarchical structure of the cluster, and is configured to include a cluster number indicating a parent cluster and a cluster number indicating a child cluster.

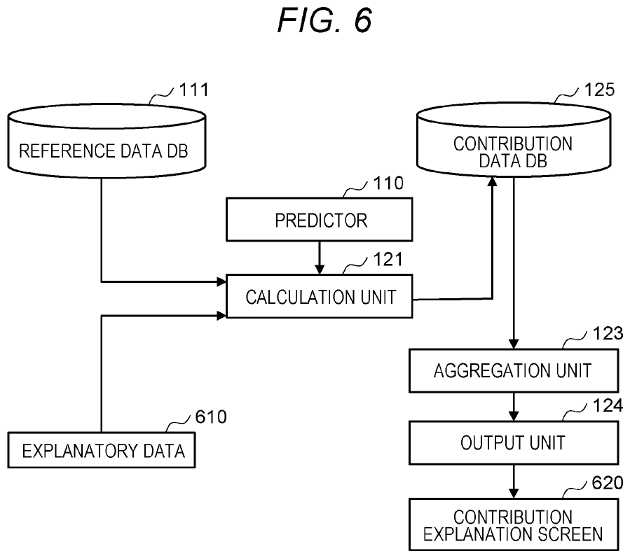

Next, a characteristic configuration of the computer system 1 will be described with reference to FIGS. 6 to 9. In the computer system 1, any of the configurations shown in FIGS. 6 to 9 and a configuration similar to the configurations can be adopted.

FIG. 6 is a diagram showing an example (a first configuration) of the characteristic configuration of the computer system 1.

The computer system 1 includes the calculation unit 121, the aggregation unit 123, and the output unit 124.

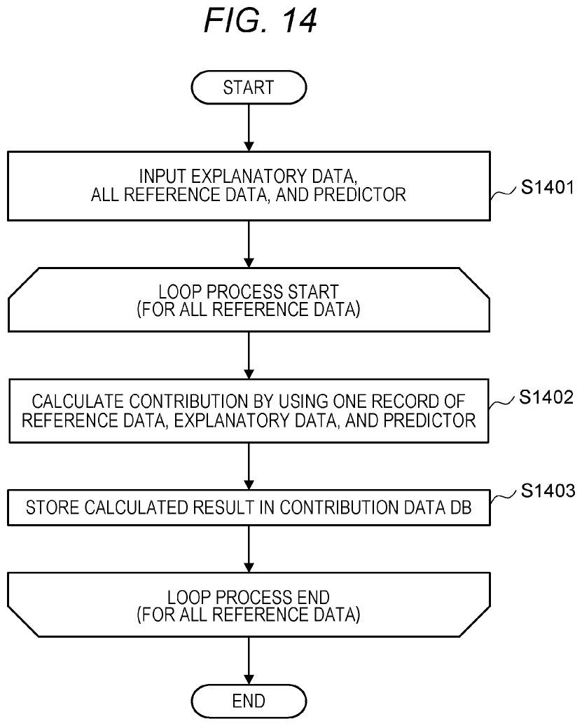

The calculation unit 121 calculates a pair contribution of each reference data and explanatory data 610 at a predetermined timing, by using the predictor 110, all the reference data in the reference data DB 111, and the explanatory data 610. The contribution data DB 125 stores the pair contribution (contribution data) that has been calculated by the calculation unit 121. It is to be noted that a process of the calculation unit 121 will be described later with reference to FIG. 14.

As an additional note, the predetermined timing may be a timing when a user gives an instruction for a prediction of the explanatory data 610 on the terminal device 101, may be a timing when the user gives an instruction for an explanation of the determination grounds after the user confirms the predicted value with respect to the explanatory data 610 on the terminal device 101, or may be another timing.

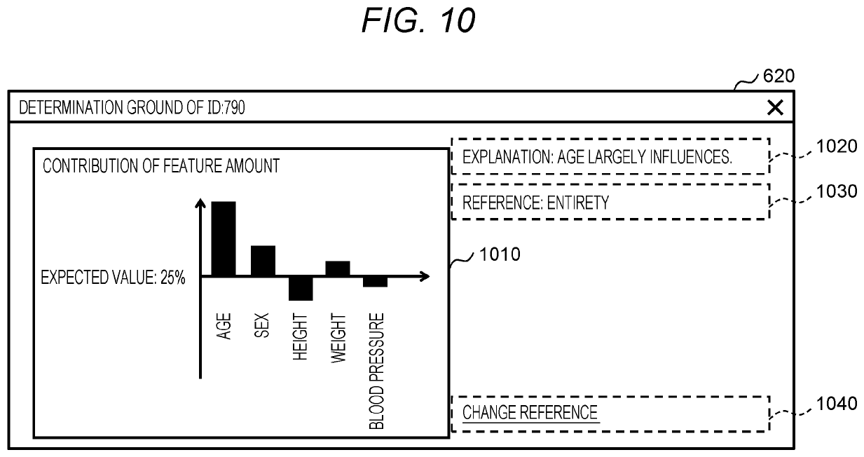

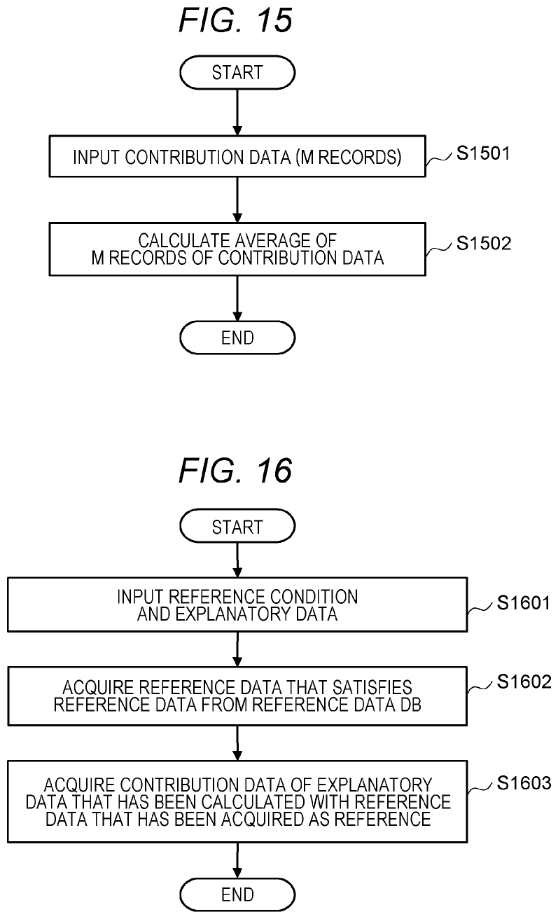

The aggregation unit 123 calculates the contribution by calculating an average of the contribution data that has been calculated by the calculation unit 121. A process of the aggregation unit 123 will be described later with reference to FIG. 15. The output unit 124 generates and outputs a contribution explanation screen 620 as a screen for explaining the contribution that has been calculated by the aggregation unit 123. The contribution explanation screen 620 will be described later with reference to FIG. 10.

In the computer system 1, the reference data DB 111 may store the explanatory data 610 as the reference data.

In the first configuration, the user can understand the contribution of each feature amount of the explanatory data 610. Further, for example, in the first configuration, the pair contribution that has been calculated using each reference data as a reference is stored in the contribution data DB 125, and the aggregation unit 123 reads the pair contribution from the contribution data DB 125 and aggregates the pair contribution. This configuration enables the contribution of each feature amount of the explanatory data to be output in a prompt manner, according to a change of the reference condition.

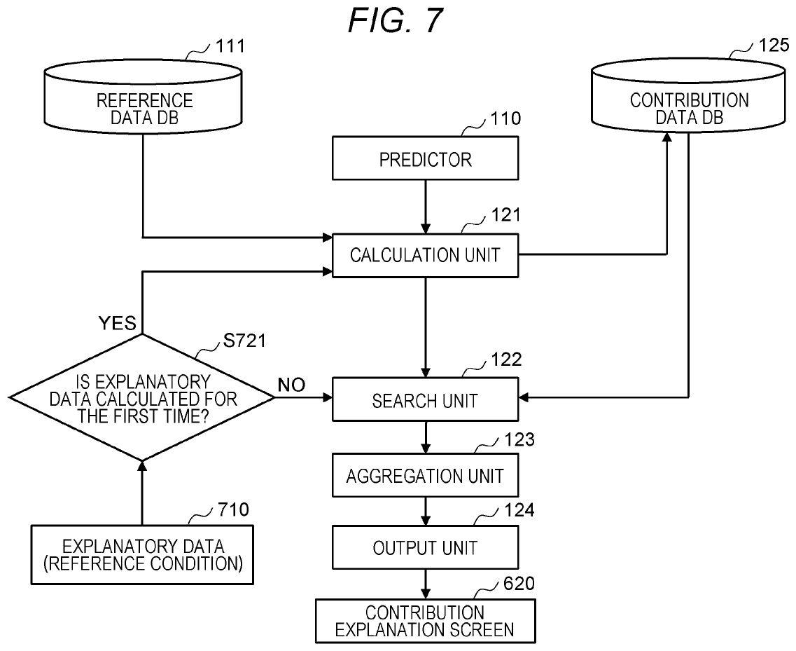

FIG. 7 is a diagram showing an example (a second configuration) of the characteristic configuration of the computer system 1. In the second configuration, the configurations different from the first configuration will be mainly described.

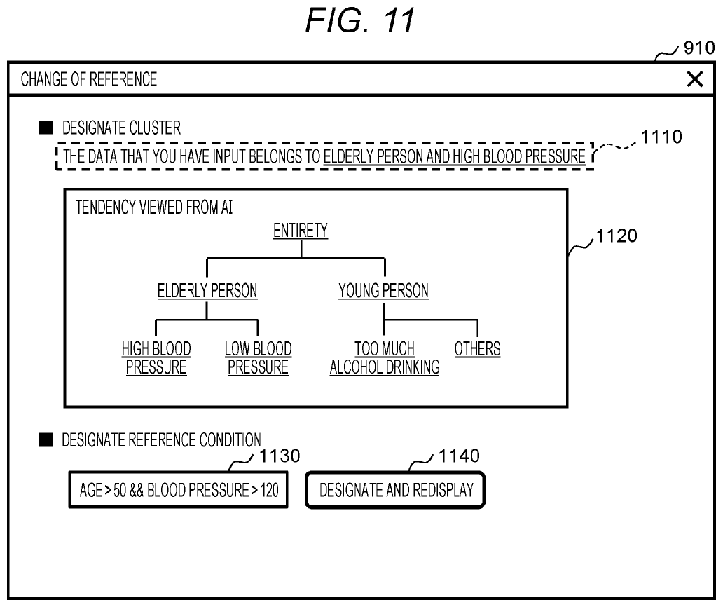

The computer system 1 further includes the search unit 122, in addition to the calculation unit 121, the aggregation unit 123, and the output unit 124. Further, in the second configuration, explanatory data (reference condition) 710 is used, instead of the explanatory data 610. The reference condition is a condition for limiting the reference data. The reference condition is set on, for example, a reference change screen shown in FIG. 11. It is to be noted that the explanatory data (reference condition) 710 includes a reference condition in some cases, or does not include the reference condition in the other cases.

In the computer system 1, it is determined whether the explanatory data (reference condition) 710 is the data to be calculated for the first time (S721). In a case where the explanatory data (reference condition) 710 is the data to be calculated for the first time, the process by the calculation unit 121 is performed. In a case where the explanatory data (reference condition) 710 is not the data to be calculated for the first time, a process by the search unit 122 is performed.

A determination method in S721 is not specified in particular. For example, a method for confirming whether the user has checked a check box for receiving an input of whether this is a prediction for the first time, at the time of estimating the explanatory data (reference condition) 710, may be used, a method for holding a history of the explanatory data (reference condition) 710 that has been predicted and confirming the history may be used, or another method may be used.

The process by the calculation unit 121 is basically the same as the process in the first configuration. However, in a case where the explanatory data (reference condition) 710 includes the reference condition, the calculation unit 121 notifies the search unit 122 of the reference condition.

The search unit 122 searches the contribution data DB 125 for the reference data that satisfies the reference condition and the contribution data that corresponds to the explanatory data (reference condition) 710. A process of the search unit 122 will be described later with reference to FIG. 16.

The aggregation unit 123 calculates the contribution by calculating the average of the contribution data that has been searched for by the search unit 122.

According to the second configuration, in a case where the explanatory data (reference condition) 710 is not the data to be calculated for the first time, the calculation by the calculation unit 121 becomes unnecessary. This configuration enables the contribution of each feature amount of the explanatory data to be obtained in a prompt manner after a change of the reference condition.

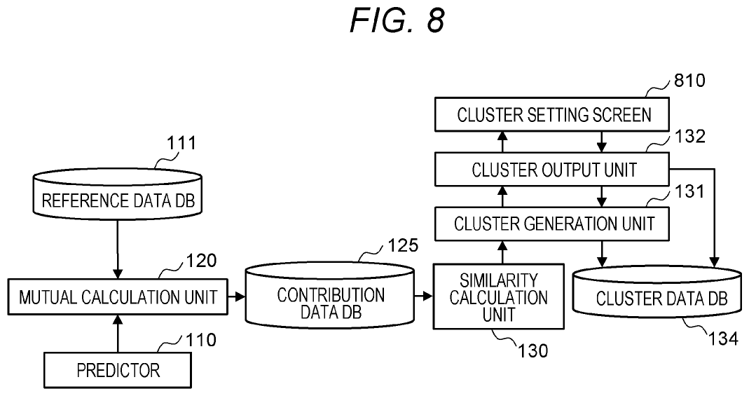

FIG. 8 is a diagram showing an example (a third configuration) of the characteristic configuration of the computer system 1.

The computer system 1 includes the mutual calculation unit 120, the similarity calculation unit 130, the cluster generation unit 131, and the cluster output unit 132.

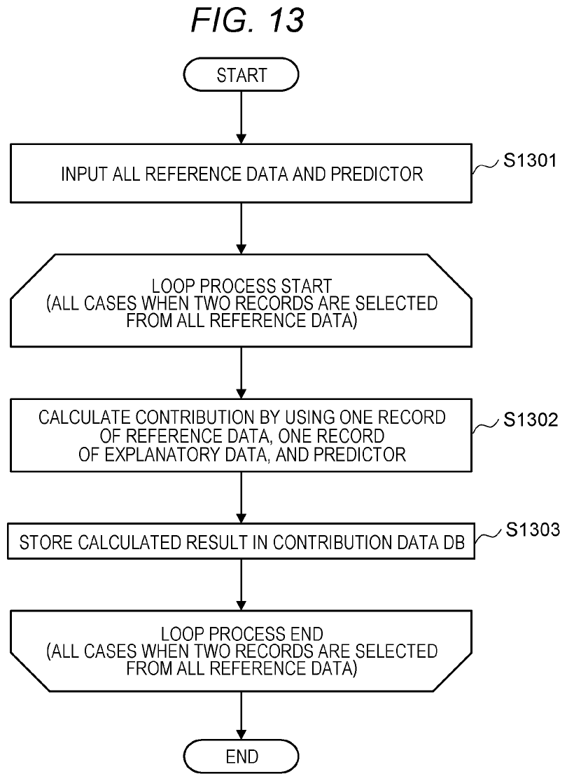

The mutual calculation unit 120 calculates a pair contribution between the reference data at a predetermined timing by using the predictor 110 and all the reference data in the reference data DB 111. The contribution data DB 125 stores the pair contribution (contribution data) that has been calculated by the mutual calculation unit 120. It is to be noted that a process of the mutual calculation unit 120 will be described later with reference to FIG. 13.

As an additional note, the predetermined timing may be a timing when the operation of the computer system 1 is started, a timing when the reference data is stored in the reference data DB 111, or another timing.

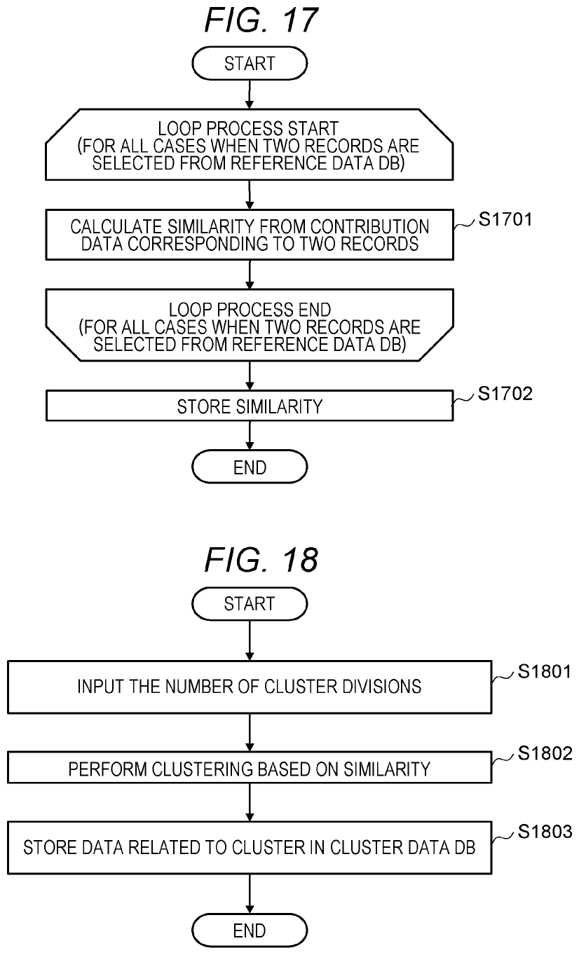

The similarity calculation unit 130 calculates the similarity between the reference data based on the contribution data DB 125. The similarity that has been calculated by the similarity calculation unit 130 is stored in the subsidiary storage device 203 in association with an explanation ID and a reference ID. It is to be noted that the contribution data DB 125 may be configured to additionally include the similarity that has been calculated by the similarity calculation unit 130. A process of the similarity calculation unit 130 will be described later with reference to FIG. 17.

The cluster generation unit 131 generates a cluster based on the similarity that has been calculated by the similarity calculation unit 130. The cluster data DB 134 stores data related to the cluster that has been generated by the cluster generation unit 131. A process of the cluster generation unit 131 will be described later with reference to FIG. 18.

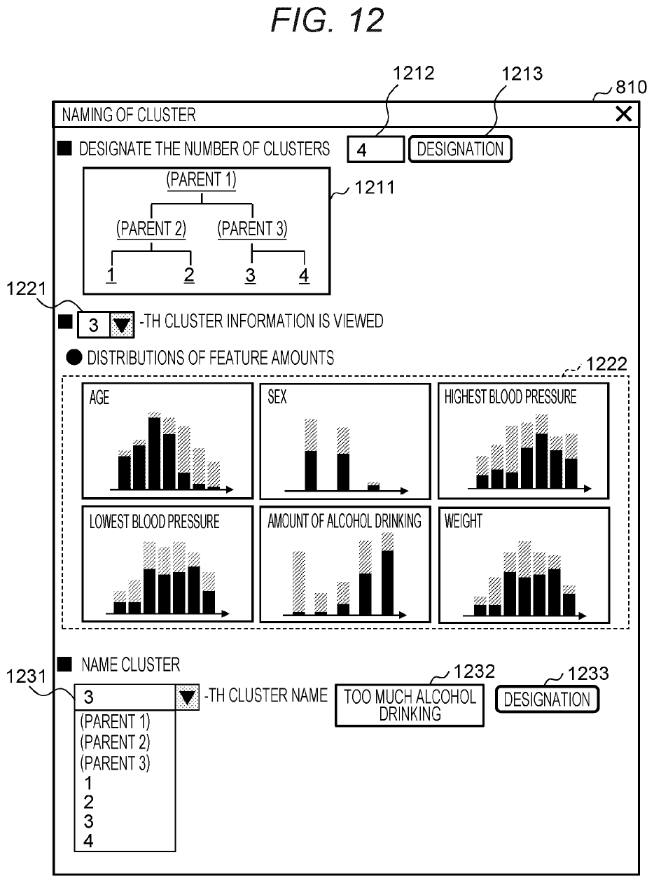

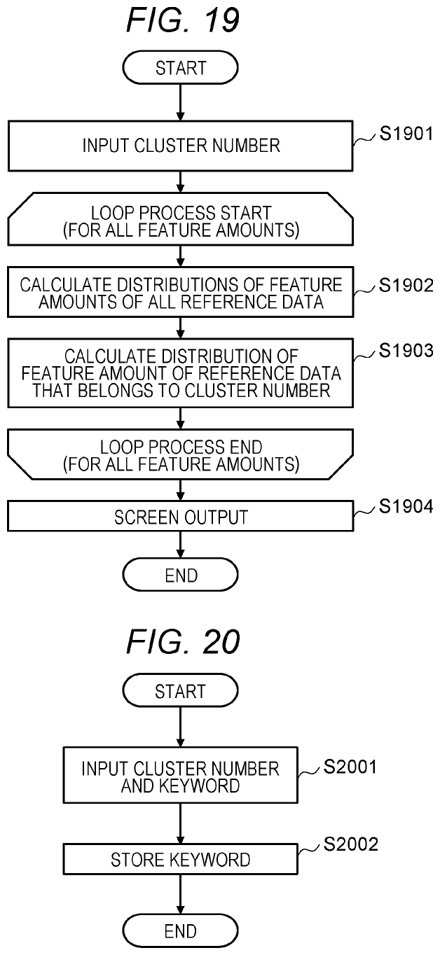

The cluster output unit 132 generates and outputs a cluster setting screen 810 as a screen for making settings related to the cluster that has been generated by the cluster generation unit 131. It is to be noted that a process of the cluster output unit 132 will be described later with reference to FIGS. 19 and 20. The cluster setting screen 810 will be described later with reference to FIG. 12.

In the third configuration, since the cluster setting screen 810 is output, for example, a system administrator is able to easily make settings related to the cluster.

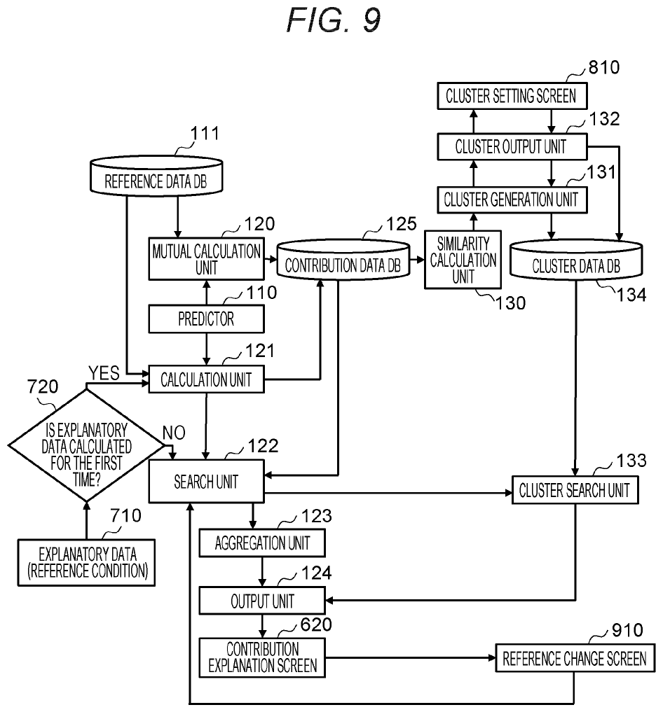

FIG. 9 is a diagram showing an example (a fourth configuration) of the characteristic configuration of the computer system 1. The fourth configuration is a configuration including the first configuration, the second configuration, and the third configuration. In the fourth configuration, configurations different from the first configuration to the third configuration will be mainly described.

The computer system 1 includes the cluster search unit 133, in addition to the mutual calculation unit 120, the calculation unit 121, the search unit 122, the aggregation unit 123, the output unit 124, the similarity calculation unit 130, the cluster generation unit 131, and the cluster output unit 132.

In a case where the explanatory data (reference condition) 710 is the data to be calculated for the first time, the similarity calculation unit 130 calculates the similarity between the explanatory data (reference condition) 710 and each of the reference data, based on the contribution data DB 125. The similarity that has been calculated by the similarity calculation unit 130 is stored in the subsidiary storage device 203 in association with an explanation ID and a reference ID.

It is to be noted that the similarity calculation may be performed for the contribution data (difference) related to the explanatory data (reference condition) 710 as described above, or may be performed for all of the contribution data (entirety) stored in the contribution data DB 125 without storing the similarity in the subsidiary storage device 203.

The search unit 122 searches the contribution data DB 125 for the contribution data, and also sends the explanation ID of the explanatory data (reference condition) 710 to the cluster search unit 133. The cluster search unit 133 refers to the cluster belonging table 510 of the cluster data DB 134, and extracts a cluster number associated with the explanation ID. The cluster search unit 133 refers to the cluster structure table 520 of the cluster data DB 134, and extracts a keyword associated with the cluster number that has been extracted. The cluster search unit 133 sends, to the output unit 124, the keyword that has been extracted.

The output unit 124 generates and outputs the contribution explanation screen 620, and also generates a reference change screen 910, which can be transitioned from the contribution explanation screen 620, and which includes the keyword that has been extracted by the cluster search unit 133. The reference change screen 910 will be described later with reference to FIG. 11.

According to the fourth configuration, the reference change screen 910 including the keyword of the cluster to which the explanatory data belongs is output. Therefore, for example, the user is able to understand the cluster to which the explanatory data belongs, and is able to easily change the reference condition.

FIG. 10 is a diagram showing an example of the contribution explanation screen 620. The contribution explanation screen 620 is displayed on the terminal device 101 operated by the user.

The contribution explanation screen 620 is a screen for displaying information related to the contribution. More specifically, the contribution explanation screen 620 includes a contribution display area 1010, an explanation display area 1020, a reference condition display area 1030, and a link display area 1040.

The contribution display area 1010 is an area for displaying the contribution of each feature amount of the explanatory data. The horizontal axis of a graph displayed in the contribution display area 1010 represents the feature amount, and the vertical axis represents the contribution. Such a graph indicates how high or low the contributions are with respect to the expected value (average of the predicted values of the reference data).

By looking at the contribution display area 1010, the user can easily understand the determination grounds for the predicted value and what feature amount and how influences the predicted value.

The explanation display area 1020 is an area for displaying main determination grounds for the predicted value. The reference condition display area 1030 is an area for displaying the reference condition. The link display area 1040 is an area for displaying a link for transitioning to the reference change screen 910 in order to change the reference condition. The user is able to display the reference change screen 910 by clicking the link in the link display area 1040.

FIG. 11 is a diagram showing an example of the reference change screen 910. The reference change screen 910 is displayed on the terminal device 101 operated by the user.

The reference change screen 910 is a screen so that the user changes the reference condition. More specifically, the reference change screen 910 is configured to include a belonging display area 1110, a cluster designation area 1120, a reference condition designation area 1130, and a change icon 1140.

The belonging display area 1110 is an area for displaying to which cluster the explanatory data that the user has input belongs. The cluster designation area 1120 is an area for receiving a change of the reference condition from a clustering result. The user confirms the belonged cluster displayed in the belonging display area 1110, and clicks a desired cluster displayed in the cluster designation area 1120, so that the user can change the reference condition.

According to the belonging display area 1110 and the cluster designation area 1120, even in a case where the user does not have specialized knowledge about the selection of the reference data, the user is able to change the reference condition appropriately. For example, in a case where the reference condition is “entirety”, the user is able to change the reference condition to “elderly person” or “elderly person and high blood pressure” so as to obtain the determination grounds based on the cluster to which the user belong. When the user clicks a cluster, an ID that belongs to the cluster is acquired, the reference data of the ID that has been acquired and the contribution data corresponding to the explanatory data are searched for, the contribution is calculated, and the contribution explanation screen 620 is displayed.

The reference condition designation area 1130 is an area for receiving an input of the reference condition. The change icon 1140 is an icon for changing the current reference condition to the reference condition that has been input into the reference condition designation area 1130. When the user inputs the reference condition in the reference condition designation area 1130 and clicks the change icon 1140, the reference data that satisfies the reference condition that has been changed and the contribution data that corresponds to the explanatory data are searched for, the contribution is calculated, and the contribution explanation screen 620 is displayed.

FIG. 12 is a diagram showing an example of the cluster setting screen 810. The cluster setting screen 810 is displayed on the terminal device 101 operated by a system administrator.

The cluster setting screen 810 is a screen for the system administrator to make settings related to the cluster. More specifically, the cluster setting screen 810 includes a cluster display area 1211, a cluster division number designation area 1212, and a designation icon 1213.

The cluster display area 1211 is an area for displaying a clustering result, based on the number of divisions that is currently set. It is to be noted that numbers “1”, “2”, “3”, and “4” displayed in the cluster display area 1211 respectively indicate the number of cluster divisions, and do not indicate cluster numbers. As an additional note, in this example, the cluster numbers are assigned such that a cluster number “1” is assigned to “parent 1”, and a cluster number “2” is assigned to “parent 2”.

The cluster division number designation area 1212 is an area for designating the number of cluster divisions. The designation icon 1213 is an icon for changing the current number of divisions to the number of divisions that has been input into the cluster division number designation area 1212. When the system administrator inputs the number of divisions in the cluster division number designation area 1212 and clicks the designation icon 1213, clustering is performed with the designated number of divisions, and the cluster setting screen 810 is updated and displayed.

In addition, the cluster setting screen 810 includes a confirmation cluster designation area 1221 and a distribution display area 1222.

In the computer system 1, a plurality of categories are provided for each feature amount of the reference data. For example, regarding age, a plurality of categories, such as 0 to 9 years old, 10 to 19 years old, and 20 to 29 years old, are provided. The confirmation cluster designation area 1221 is an area for designating the cluster that the system administrator intends to confirm the number of reference data that belong to respective categories of the feature amounts (distributions of the feature amounts), when the system administrator sets a name for each cluster.

The distribution display area 1222 is an area for displaying the distribution of each feature amount in the cluster that has been designated in the confirmation cluster designation area 1221. A filled bar graph displayed in the distribution display area 1222 indicates the number of reference data that belong to the designated cluster, whereas a shaded bar graph indicates the number of all the reference data.

When the cluster designation is changed in the confirmation cluster designation area 1221, the ID that belongs to the cluster that has been changed is specified based on the cluster belonging table 510, the reference data of the ID that has been specified is extracted from the reference data DB 111, a distribution of each feature amount is calculated from the reference data that has been extracted, and the distribution display area 1222 is displayed.

According to the distribution display area 1222, the system administrator can easily understand a tendency of the cluster that has been designated in the confirmation cluster designation area 1221, when compared with the entirety.

Further, the cluster setting screen 810 includes a naming cluster designation area 1231, a cluster name input area 1232, and a designation icon 1233.

The naming cluster designation area 1231 is an area for the system administrator to designate the cluster, in intending to set a name of the cluster. The cluster name input area 1232 is an area for the system administrator to input the name of the cluster. The designation icon 1233 is an icon for the system administrator to set the name that has been input into the cluster name input area 1232 to the cluster that has been designated in the naming cluster designation area 1231. When the designation icon 1233 is clicked, the name that has been input into the cluster name input area 1232 is registered in the cluster structure table 520, in a keyword of the cluster number of the cluster that has been designated in the naming cluster designation area 1231.

The cluster setting screen 810 is capable of assisting the system administrator to set a human-understandable name to the cluster.

FIG. 13 is a diagram showing an example of a flowchart related to a process performed by the mutual calculation unit 120.

In S1301, the mutual calculation unit 120 acquires, as inputs, all the reference data stored in the reference data DB 111 and the predictor 110.

The mutual calculation unit 120 performs processes of S1302 and S1303 for all cases (all pairs), when two records are selected from all the reference data.

In S1302, the mutual calculation unit 120 sets one of the two records of the reference data that have been selected to the explanatory data (selected explanatory data) and the other one to the reference data (selected reference data), and calculates the contribution of each feature amount of the selected explanatory data by using the predictor 110.

For example, the mutual calculation unit 120 perturbates each feature amount of the selected explanatory data by using the selected reference data, and generates a plurality of synthetic data. The perturbation here means that, for example, a part of the selected explanatory data is changed to a feature amount of the selected reference data a plurality of times, such that the values of the selected explanatory data are used for age and gender, and the other features are changed to the features of the selected reference data. The plurality of times may be the number of the synthetic data of all conceivable cases, or may be less than or equal to the number of the synthetic data of all conceivable cases. The mutual calculation unit 120 obtains a predicted value for each of the plurality of synthetic data, by using the predictor 110. In this situation, the mutual calculation unit 120 calculates a difference in the predicted values generated by the perturbation with respect to each feature amount of the selected explanatory data, and calculates a weighted average of the difference as a contribution.

In S1303, the mutual calculation unit 120 stores the contribution that has been calculated as a pair contribution (contribution data) in the contribution data DB 125.

FIG. 14 is a diagram showing an example of a flowchart related to a process performed by the calculation unit 121.

In S1401, the calculation unit 121 acquires, as inputs, the explanatory data, all the reference data stored in the reference data DB 111, and the predictor 110.

The calculation unit 121 performs processes S1402 and S1403 with respect to all the reference data.

In S1402, the calculation unit 121 calculates a contribution of each feature amount of the explanatory data, by using one record of the reference data, the explanatory data, and the predictor 110. It is to be noted that the calculation method is the same as that of S1302.

In S1403, the calculation unit 121 stores the contribution that has been calculated, as a pair contribution (contribution data) in the contribution data DB 125.

FIG. 15 is a diagram showing an example of a flowchart related to a process performed by the aggregation unit 123.

In the S1501, in a case where the contribution data that has been calculated by the calculation unit 121 or the contribution data that has been searched for by the search unit 122 is M records, the aggregation unit 123 receives the M records of the contribution data, as inputs.

In S1502, the aggregation unit 123 calculates the average of the M records of the contribution data. For example, in a case where three records of the contribution data are “age: 0.5, gender: 0.02, . . . ”, “age: 0.7, gender: 0.04, . . . ”, and “age: 0.6, gender: 0.03, . . . ”, the aggregation unit 123 calculates “age: 0.6 (=(0.5+0.7+0.6)/3), gender: 0.03 (=(0.02+0.04+0.03)/3), . . . ”.

FIG. 16 is a diagram showing an example of a flowchart related to a process performed by the search unit 122.

In S1601, the search unit 122 acquires, as inputs, the reference condition and the explanatory data.

In S1602, the search unit 122 searches the reference data DB 111 for the reference data that satisfies the reference condition, and acquires an ID of the reference data that has been searched for.

In S1603, the search unit 122 searches the contribution data DB 125 for the contribution data of the explanatory data that has been calculated with the reference data of the ID that has been acquired as a reference, and acquires the contribution data that has been searched for.

FIG. 17 is a diagram showing an example of a flowchart related to a process performed by the similarity calculation unit 130.

The similarity calculation unit 130 performs a process of S1701 for all cases, when two records are selected from all the reference data in the reference data DB 111.

In S1701, the similarity calculation unit 130 calculates a similarity of the two records of the reference data that has been selected. More specifically, the similarity calculation unit 130 extracts the contribution data (contribution vector) corresponding to the two records of the reference data from the contribution data DB 125, and calculates a similarity from the contribution vector that has been extracted by a function for calculating an optional similarity (similarity calculation function). For example, in a case where the similarity calculation function is a function for finding the length of a vector, the similarity calculation unit 130 calculates the length of an n-dimensional contribution vector in L(x)=(x₁²+ . . . +xₙ²)^{1/2}.

In S1702, the similarity calculation unit 130 stores the similarity that has been calculated in the subsidiary storage device 203 in association with the IDs of the two records of the reference data that has been selected.

FIG. 18 is a diagram showing an example of a flowchart related to a process performed by the cluster generation unit 131.

In S1801, the cluster generation unit 131 acquires the number of the cluster divisions as an input. The cluster generation unit 131 acquires the number of the cluster divisions in a case where the number of the cluster divisions is set on the cluster setting screen 810, and acquires a default number of the cluster divisions in a case where the number of the cluster divisions is not set on the cluster setting screen 810.

In S1802, the cluster generation unit 131 performs clustering based on the similarity stored in the subsidiary storage device 203. For example, the cluster generation unit 131 generates a tree diagram based on the similarity stored in the subsidiary storage device 203, and cuts the tree diagram at a point corresponding to the number of the cluster divisions that has been acquired (an element connected below is treated as one cluster).

In S1803, the cluster generation unit 131 stores, in the cluster data DB 134, the data related to the cluster that has been generated.

FIG. 19 is a diagram showing an example of a flowchart related to a process performed by the cluster output unit 132.

In S1901, the cluster output unit 132 acquires, as an input, cluster information (cluster number) that has been designated in the confirmation cluster designation area 1221 on the cluster setting screen 810.

The cluster output unit 132 performs processes S1902 and S1903 for all feature amounts of the reference data.

In S1902, the cluster output unit 132 calculates distributions of all the reference data (total number of the records for each category) for the feature amount to be processed.

In S1903, the cluster output unit 132 calculates the distribution of the reference data that belongs to the cluster number acquired in S1901 (total number of the records for each category) for the feature amount to be processed.

In S1904, the cluster output unit 132 updates the distribution display area 1222 on the cluster setting screen 810, based on the distributions calculated in S1902 and S1903, and sends the distribution display area 1222 that has been updated to the terminal device 101.

FIG. 20 is a diagram showing an example of a flowchart related to a process performed by the cluster output unit 132.

In S2001, the cluster output unit 132 acquires, as inputs, the cluster information (cluster number) designated in the naming cluster designation area 1231 on the cluster setting screen 810 and the name (keyword) that has been input into the cluster name input area 1232.

In S2002, the cluster output unit 132 stores, in the cluster structure table 520, the name that has been acquired in the keyword that corresponds to the cluster number that has been acquired.

According to embodiments of the present embodiment, it is possible to provide a computer system that is high in convenience.

### (2) Additional Notes

The above embodiment includes, for example, the following contents.

In the above-described embodiment, the case where the present invention is applied to a computer system has been described. However, the present invention is not limited to this, and can be widely applied to various other systems, devices, methods, and programs.

Further, in the above-described embodiment, the reference data has been described with reference to FIG. 3 as an example. However, the present invention is not limited to this, and the reference data may be image data, audio data, or other data.

Further, in the above-described embodiment, the configuration of each table is an example. One table may be divided into two or more tables, or all or a part of the two or more tables may be integrated into one table.

Further, in the above-described embodiment, various types of data have been described using an XX table for convenience of description. However, the data structure is not limited, and may be represented as XX information or the like.

Further, in the above-described embodiment, the case where an average value is used as a statistical value has been described. However, the statistical value is not limited to the average value, and may be another statistical value such as a maximum value, a minimum value, a difference between the maximum value and the minimum value, and a most frequent value, a median, or a standard deviation.

Further, in the above-described embodiment, an output of information is not limited to displaying on a display. The output of the information may be an audio output by a speaker, an output to a file, printing on a paper medium or the like by a printing device, projection on a screen or the like by a projector, or another form.

Further, the screens displayed in the above-described embodiment are examples, and any screen design may be used as long as the received information is the same.

Further, in the above description, information such as programs, tables, and files for realizing respective functions is stored in a memory, a hard disk, a storage device such as an SSD (Solid State Drive) or a recording medium such as an IC card, an SD card, or a DVD.

The embodiment described above has, for example, the following characteristic configurations.

A computer system (for example, the computer system 1) that uses a predictor (the predictor 110) configured to conduct a prediction, explanatory data (for example, the explanatory data 610, the explanatory data (reference condition) 710) that is data to be a prediction target of the predictor, and a plurality of pieces of reference data (for example, a part or the entire of the reference data stored in the reference data DB 111) that are data to be used as a reference in comparison with the explanatory data, and that calculates a contribution of each feature amount of the explanatory data with respect to a predicted value of the explanatory data that has been predicted by the predictor, the computer system including: a calculation unit (for example, the calculation unit 121, the computer 100-2, the computer 100, or another computer or circuit) configured to extract one piece of the reference data from the plurality of pieces of reference data, configured to calculate the contribution of each feature amount of the explanatory data with respect to the predicted value by using the one piece of the reference data, the explanatory data, and the predictor, and configured to store, in a storage device (for example, the subsidiary storage device 203, the computer 100-2, the computer 100, or another computer), the contribution that has been calculated as a pair contribution in association with the one piece of the reference data and the explanatory data, the pair contribution being a contribution that has been calculated with the one piece of the reference data and the explanatory data being a pair, for all pairs including each reference data of the plurality of pieces of reference data and the explanatory data (for example, see FIG. 14); and

an aggregation unit (for example, the aggregation unit 123, the computer 100-2, the computer 100, or another computer or circuit) configured to read, from the storage device, the pair contribution that has been calculated by the calculation unit for each feature amount of the explanatory data, and configured to calculate by aggregating the contribution of each feature amount of the explanatory data (for example, see FIG. 15).

In the above configuration, the pair contribution that has been calculated with each reference data as a reference is stored in the storage device. For example, the computer system includes the display unit that displays the contribution that has been aggregated by the aggregation unit, so that the user can understand the contribution of each feature amount of the explanatory data. Further, for example, the computer system includes spreadsheet software, so that the user can aggregate the pair contribution stored in the storage device using the spreadsheet software, and therefore can understand the contribution of each feature amount of the explanatory data.

Further, in the above configuration, for example, the aggregation unit is capable of reading the pair contribution from the storage device and aggregating the pair contribution. Therefore, the contribution of each feature amount of the explanatory data can be output in a prompt manner, according to a change of the reference condition. The reference condition may be designated by a user (designated with the cluster or designated by inputting the reference condition), or may be automatically set from the explanatory data (one or a plurality of categories to which one or a plurality of feature amounts belong may be set such that, for example, the age is equal to or older than 50 and equal to or younger than 59 years old, and in addition, the weight is equal to or more than 70 kg and equal to or less than 79 kg).

The above computer system further includes a terminal device (for example, the terminal device 101) configured to input a reference condition, a search unit (for example, the search unit 122, the computer 100-2, the computer 100, or another computer or circuit) configured to search the storage device for the pair contribution corresponding to reference data that satisfies the reference condition that has been input on the terminal device from among the plurality of pieces of reference data and the explanatory data (for example, see FIG. 16), and an output unit (for example, the output unit 124, the computer 100-2, the computer 100, or another computer or circuit) configured to output, to the terminal device, information indicating the contribution of the each feature amount of the explanatory data that has been calculated by the aggregation unit aggregating the pair contribution that has been searched for by the search unit, for the each feature amount of the explanatory data.

In the above configuration, for example, when a reference condition is input on the terminal device, the pair contribution corresponding to the reference data that satisfies the reference condition is searched for and aggregated, and the contribution of each feature amount of the explanatory data corresponding to the reference condition is output. According to the above configuration, the calculation by the calculation unit becomes unnecessary. Therefore, the contribution of each feature amount of the explanatory data after the reference condition is changed can be obtained in a prompt manner.

The above computer system further includes: a mutual calculation unit (for example, the mutual calculation unit 120, the computer 100-2, the computer 100, or another computer or circuit) configured to extract a pair of two pieces of reference data from the plurality of pieces of reference data, configured to set one of the pair of the two pieces of reference data that has been extracted to a first reference data and the other one of the pair to a first explanatory data, configured to calculate a contribution of each feature amount of the first explanatory data with respect to the predicted value by using the first reference data, the first explanatory data, and the predictor, and configured to store, in the storage device, the contribution that has been calculated as the pair contribution in association with the first reference data and the first explanatory data, the pair contribution being a contribution that has been calculated with the first reference data and the first explanatory data being a pair, for all pairs of the plurality of reference data (for example, see FIG. 13); a similarity calculation unit (for example, the similarity calculation unit 130, the computer 100-3, the computer 100, or another computer or circuit) configured to calculate a similarity between data in association with each pair contribution, by using the each pair contribution, for the each pair contribution stored in the storage device (for example, see FIG. 17); a cluster generation unit (for example, the cluster generation unit 131, the computer 100-3, the computer 100, or another computer or circuit) configured to generate a cluster based on the similarity that has been calculated by the similarity calculation unit (for example, see FIG. 18); and a cluster output unit (for example, the cluster output unit 132, the computer 100-3, the computer 100, or another computer or circuit) configured to output information indicating the cluster that has been generated by the cluster generation unit (for example, see FIGS. 19 and 20).

In the above configuration, since the cluster is generated and output, for example, a system administrator is able to easily make settings related to the cluster.

The above computer system further includes a terminal device (for example, the terminal device 101) on which the cluster that has been generated by the cluster generation unit is selectable, a search unit (for example, the search unit 122, the computer 100-2, the computer 100, or another computer or circuit) configured to search the storage device for the pair contribution corresponding to reference data that belongs to the cluster that has been selected on the terminal device and the explanatory data, and an output unit (for example, the output unit 124, the computer 100-2, the computer 100, or another computer or circuit) configured to generate screen information and send the screen information to the terminal device, the screen information indicating the contribution of the each feature amount of the explanatory data that has been calculated by the aggregation unit aggregating the pair contribution that has been searched for by the search unit, for the each feature amount of the explanatory data.

In the above configuration, for example, a user is able to change the reference condition by designating the cluster. According to the above configuration, even in a case where the user does not know how to change the reference condition, the user is able to change the reference condition appropriately and is able to understand the contribution of each feature amount of the explanatory data after the reference condition is changed.

The above-described computer system further includes a terminal device (for example, the terminal device 101) configured to input the explanatory data, and an output unit (for example, the output unit 124, the computer 100-2, the computer 100, or another computer or circuit) configured to send, to the terminal device, information indicating the contribution of the each feature amount of the explanatory data that has been aggregated by the aggregation unit.

In the above configuration, for example, since the contribution of each feature amount of the explanatory data is output on the terminal device, the user who has obtained the predicted value of the explanatory data is able to understand the determination grounds for the predicted value.

In addition, the configurations described above may be appropriately changed, recombined, combined, or omitted without departing from the scope of the present invention.

What is claimed is:

1. A computer system that uses a predictor configured to conduct a prediction, explanatory data that is data to be a prediction target of the predictor, and a plurality of pieces of reference data that are data to be used as a reference in comparison with the explanatory data, and that calculates a contribution of each feature amount of the explanatory data with respect to a predicted value of the explanatory data that has been predicted by the predictor, the computer system comprising:
a calculation unit configured to extract one piece of the reference data from the plurality of pieces of reference data, configured to calculate the contribution of the each feature amount of the explanatory data with respect to the predicted value by using the one piece of the reference data, the explanatory data, and the predictor, and configured to store, in a storage device, the contribution that has been calculated as a pair contribution in association with the one piece of the reference data and the explanatory data, the pair contribution being a contribution that has been calculated with the one piece of the reference data and the explanatory data being a pair, for all pairs including each reference data of the plurality of pieces of reference data and the explanatory data; and
an aggregation unit configured to read, from the storage device, the pair contribution that has been calculated by the calculation unit for the each feature amount of the explanatory data, and configured to calculate by aggregating the contribution of the each feature amount of the explanatory data.

2. The computer system according to claim 1, further comprising:
a terminal device configured to input a reference condition;
a search unit configured to search the storage device for the pair contribution corresponding to reference data that satisfies the reference condition that has been input on the terminal device from the plurality of pieces of reference data and the explanatory data; and
an output unit configured to output, to the terminal device, information indicating the contribution of the each feature amount of the explanatory data that has been calculated by the aggregation unit aggregating the pair contribution that has been searched for by the search unit for the each feature amount of the explanatory data.

3. The computer system according to claim 1, further comprising:
a mutual calculation unit configured to extract a pair of two pieces of reference data from the plurality of pieces of reference data, configured to set one of the pair of the two pieces of reference data that has been extracted to a first reference data and the other one of the pair to a first explanatory data, configured to calculate a contribution of each feature amount of the first explanatory data with respect to the predicted value by using the first reference data, the first explanatory data, and the predictor, and configured to store, in the storage device, the contribution that has been calculated as the pair contribution in association with the first reference data and the first explanatory data, the pair contribution being a contribution that has been calculated with the first reference data and the first explanatory data being a pair, for all pairs of the plurality of reference data;
a similarity calculation unit configured to calculate a similarity between data in association with each pair contribution, by using the each pair contribution, for the each pair contribution stored in the storage device;
a cluster generation unit configured to generate a cluster based on the similarity that has been calculated by the similarity calculation unit; and
a cluster output unit configured to output information indicating the cluster that has been generated by the cluster generation unit.

4. The computer system according to claim 3, further comprising:
a terminal device on which the cluster that has been generated by the cluster generation unit is selectable;
a search unit configured to search the storage device for the pair contribution corresponding to reference data that belongs to the cluster that has been selected on the terminal device and the explanatory data; and
an output unit configured to generate screen information and send the screen information to the terminal device, the screen information indicating the contribution of the each feature amount of the explanatory data that has been calculated by the aggregation unit aggregating the pair contribution that has been searched for by the search unit, for the each feature amount of the explanatory data.

5. The computer system according to claim 1, further comprising:
a terminal device configured to input the explanatory data; and
an output unit configured to send, to the terminal device, information indicating the contribution of the each feature amount of the explanatory data that has been aggregated by the aggregation unit.

6. A contribution calculation method in a computer system that uses a predictor configured to conduct a prediction, explanatory data that is data to be a prediction target of the predictor, and a plurality of pieces of reference data that are data to be used as a reference in comparison with the explanatory data, and that calculates a contribution of each feature amount of the explanatory data with respect to a predicted value of the explanatory data that has been predicted by the predictor, the contribution calculation method comprising:
extracting, by a calculation unit included in the computer system, one piece of the reference data from the plurality of pieces of reference data, calculating the contribution of the each feature amount of the explanatory data with respect to the predicted value by using the one piece of the reference data, the explanatory data, and the predictor, and storing, in a storage device, the contribution that has been calculated as a pair contribution in association with the one piece of the reference data and the explanatory data, the pair contribution being a contribution that has been calculated with the one piece of the reference data and the explanatory data being a pair, for all pairs including each reference data of the plurality of pieces of reference data and the explanatory data; and
reading, by an aggregation unit included in the computer system, from the storage device, the pair contribution that has been calculated by the calculation unit for the each feature amount of the explanatory data, and configured to calculate by aggregating the contribution of the each feature amount of the explanatory data.
